(19) United States
(12) Patent Application Publication
(10) Pub. No.: US 2013/0196340 A1
(43) Pub. Date: Aug. 1, 2013

(54) EX VIVO PLASMA ENZYME ACTIVITY ASSAY USING INHIBITORS AS A NEGATIVE CONTROL

(71) Applicant: ROAR HOLDING LLC, New York, NY (US)

(72) Inventor: Robert W. BROCIA, Bronxville, NY (US)

(73) Assignee: ROAR HOLDING LLC, New York, NY (US)

(21) Appl. No.: 13/754,429

(22) Filed: Jan. 30, 2013

Related U.S. Application Data
(60) Provisional application No. 61/592,534, filed on Jan. 30, 2012.

(51) Int. Cl. G01N 33/68

(52) U.S. Cl. CPC G01N 33/68
USPC 435/7.1

(57) ABSTRACT

An improved assay for enzyme activity in bodily fluids which permits the influence of other components of the fluid to be accounted for is improved by using a negative control where the enzyme is inactivated or bound.

### CROSS REFERENCE TO RELATED APPLICATIONS

This application claims benefit under 35 U.S.C. §119(e) to provisional application 61/592,534 filed 30 Jan. 2012. The content of the above patent application is incorporated by reference herein in its entirety.

### TECHNICAL FIELD

1. Field of the Invention

The invention is in the field of diagnostic assays, in particular the assessment of enzyme activity, for example, cholesteryl ester transfer protein (CETP) activity, in bodily fluids.

The invention is an improved method to determine enzyme activity in a sample of bodily fluid from a test subject without dilution of the sample. Dilution of the sample disrupts the physiological concentration of other components in the sample. These components may have an effect on the enzyme activity directly or indirectly.

2. Background Art

It is understood that the activity of various enzymes in bodily fluids may be altered by other components of the sample. For example, for CETP, such components present in the sample may include lipoproteins such as HDL, LDL, VLDL, IDL or apoproteins, such as C1, C2, C3 or any apolipoprotein present in such fluids.

Generally speaking, assays for measuring enzyme activity in bodily fluids are performed in diluted samples where the bodily fluid represents only a small percentage of the total reaction mixture. Provided suitable substrates are available, however, it would be preferable to conduct such assays at high-concentration levels of bodily fluid to account for interfering activities of other components contained therein. Besides CETP, a number of other enzymes are obvious candidates for such assays, including phospholipid transfer protein (PLTP), lipoprotein lipase (LPL), hepatic lipase (HL), hormone sensitive lipase (HSL), endothelial lipase (EL), phospholipase (PL), and lecithin:cholesterol acyl transferase (LCAT).

Other enzymes with established clinical relevance to which the invention may be applied include creatine phosphokinase (CPK) which catalyzes the transfer of phosphate groups between creatine and phosphocreatine and ATP and ADP, which is used as a marker for myocardial infarction; gamma glutamyl transpeptidase (GGT) which catalyzes the transfer of glutamyl groups among polypeptides and amino acids which is associated with biliary tract cancers; lactic dehydrogenase (LDH) which catalyzes the redox reaction between pyruvic and lactic acids, wherein levels of its various isoenzymes are associated with different diseases; lipase, associated with pancreatitis; and transaminases such as glutamic oxaloacetic transaminase (GOT) and glutamic pyruvic transaminase (GPT). These are among many enzymes whose levels in blood or plasma are altered according to various disease states in subjects.

Using CETP as an example, there are well-accepted methods to measure CETP activity in plasma or serum of a subject wherein the sample is added at a volume less than 10% of the total assay volume to a mixture of assay reagents plus buffer. These methods reliably measure CETP activity in the diluted sample resulting in a CETP activity value that correlates with CETP mass. This value is not necessarily related to the actual CETP activity present in the subject's blood, i.e., the value that would be measured when all of the sample components are at physiological concentration.

Plasma CETP activity may be used to monitor the efficacy of a drug used to raise HDL via inhibition of plasma CETP activity. The initial CETP activity is compared to activity after subsequent dosing with a CETP inhibitor and each subject's plasma acts as its own control. Thus any matrix effects associated with a particular plasma remains constant. However, to measure for plasma CETP activity in samples per se, the invention method accounts for the matrix effects of the undiluted sample.

Similarly, for other enzyme activities, in some cases, activity over a time course may be performed in reaction mixtures with diluted samples where the matrix effects associated with a particular plasma remain constant or are not observed. However, as was the case for CETP in order to measure the activity in samples per se, the invention method described below accounts not only for interfering or modulating substances in the bodily fluid but also the matrix effects on fluorescence measurement.

The present inventor has described CETP assays using more concentrated forms of the reaction mixture wherein the bodily fluid constitutes, for example, at least about 89% v/v of the reaction mixture in U.S. Pat. No. 7,279,297. However, in the assays described, a simple buffer solution was used as control. It has now been found that improved results for CETP and other enzymes are obtained by employing, as a negative control, a duplicate reaction mixture, but with the addition of an effective amount of an inhibitor for the enzyme. Thus the matrix effects of the sample components are canceled out.

### DISCLOSURE OF THE INVENTION

The assay method described herein demonstrates improved results in assays that account for interfering factors in the biological sample of bodily fluid being tested by using undiluted samples. This improvement is effected by having the bodily fluid present in similar concentration in the control as in the test sample and inactivating the enzyme to be assayed in the negative control.

Thus, in one aspect, the invention is directed to an improved method for detecting the level activity of an enzyme in a bodily fluid of a subject by measuring conversion of substrate to product in a reaction mixture comprising an undiluted amount of the bodily fluid, wherein the improvement comprises employing as a negative control a similar reaction mixture which further contains an inhibitor that is a binding or inactivating agent for said enzyme.

There are a number of enzymes wherein the conversion of substrate to product can be assessed by measuring the transfer of label from a donor substrate for the enzyme to an acceptor. These enzymes include cholesteryl ester transfer protein (CETP), phospholipid transfer protein (PLTP), lipoprotein lipase (LPL), hepatic lipase (HL), hormone sensitive lipase (HSL), endothelial lipase (EL), phospholipase (PL), and lecithin:cholesterol acyl transferase (LCAT).

Other enzymes for which the invention is suitable include CPK, GGT, LDH, lipase, GOP and GPT as mentioned above.

In another aspect, enzyme activity may be measured by determining the amount of inhibitor necessary to neutralize the activity in the sample. In this aspect, the assay is conducted in a manner similar to that used to measure enzyme activity described above, but serial dilutions of the inhibitor are added to multiple samples. The amount of inhibitor in the dilution required to diminish the activity can then be determined by suitable analysis of the serial dilutions.

In another aspect, the invention is directed to kits for carrying out the improved method.

### MODES OF CARRYING OUT THE INVENTION

The present invention takes account of physiological substances and conditions that affect the real enzyme activity experienced in biological fluid as it exists in the subject. The invention accomplishes this by permitting substantially undiluted samples of plasma or serum or other fluid, such as semen, urine, CSF, or saliva to be used. However, by using undiluted samples, there is often present a matrix effect which is a non-specific effect on the level of fluorescence measured simply by virtue of components of the sample that affect the transmission of fluorescence. The matrix effect can be canceled out by using as a “blank” a similar biological fluid sample which contains an effective inhibitor of the enzyme being measured. That is, the improvement canceling out the matrix effect is accomplished by inhibiting the activity of the enzyme by treatment with an inhibitor that binds or inactivates the enzyme in a control sample of said plasma or serum or other fluid.

The sample with the inhibitor acts as the negative control since the enzyme activity has been neutralized. The neutralized sample is a more appropriate assay blank than buffer, for example, because any matrix effects occurring in the sample are subtracted out when the signal, such as fluorescence intensity, of the neutralized control is subtracted from the signal of the active sample. The result is a more accurate measurement of transfer than utilizing a buffer blank as a negative control. In short, unusual spectral properties associated with the sample of bodily fluid are subtracted out.

Substantially undiluted samples have been used previously to measure CETP activity in bodily fluids. Specifically, U.S. Pat. No. 7,279,297, incorporated herein by reference, describes one such method in detail. In this particular method, CETP, which transfers neutral lipids that are cholesteryl or triglyceride esters from one particle, the donor, to another lipoprotein particle, the acceptor, is assayed. A fluorescent molecule is present in the donor in a quenched form. This is prepared by providing a donor that comprises a fluorescently labeled cholesteryl or triglyceride ester and at least phospholipid as a sonicated particle. An emulsion that contains a lipid that acts as an acceptor is formed. As the fluorescent label is confined to a particle in the donor but not in the acceptor, the fluorescence is increased upon incubation in the presence of CETP, which liberates the ester from the donor particle. The fluorescent label used in the exemplified assay is N-(7-nitrobenz-2-oxa-1,3-diazol-4-yl)amine (NBD) but, of course, other fluorescent labels could also be used, such as rhodamine, 5-butyl-4,4-difluoro-4-bora-3A,4A diaza-S-indacen (BODIPY® from Molecular Probes, Inc. Many other fluorophores are commercially available, e.g., from Sigma/Aldrich, St. Louis, Mo.

According to the above cited '297 patent, suitable acceptors are lipoprotein particles, including Intralipid® and acceptors prepared from fresh human plasma. Other descriptions of donors and acceptors useful in CETP assays are described in U.S. Pat. No. 5,535,235; U.S. Pat. No. 5,618,683; U.S. Pat. No. 5,770,355; and U.S. Pat. No. 6,974,676. The '297 patent gives a detailed description of one embodiment of a method of preparation of donor and acceptor moieties.

This specific approach which employs transfer of a label from a donor substrate to an acceptor is also applicable to the above-mentioned PLTP, LPL, HL, HSL, EL, PL, and LCAT. The invention, however, is not limited to assessment of enzymic activity that depends on transfer of a quenched fluorescent label to an acceptor where the label is not quenched. Any enzymic assay which measures conversion of substrate to product can also be employed. The progress of the reaction can be followed by any number of methods, including removing aliquots for assay using various chromatographic techniques, immunological techniques, transfer of radioactive label, and the like. The importance of canceling out a matrix effect, however, will vary with the nature of the assay. It is especially important in spectrophotometric based assays whether colorimetric or fluorescence based. Simply put, the assay for enzyme activity as previously, perhaps, conducted using a diluted sample is instead performed using an undiluted amount of sample and supplying an inhibitor of the enzyme activity to a comparable reaction mixture as a negative control.

As defined in this application, an “undiluted amount” refers to a condition wherein at least 50%-95% v/v of the reaction mixture used in the assay is the bodily fluid. The “undiluted amount” refers also to intermediate percentages between 50% and 95% as if specifically named. Thus, for example, at least 60%, 70%, 80%, 85%, 90%, etc., of bodily fluid may be present in the reaction mixture. The test sample and negative control are identical except that an inhibitor for neutralizing the enzyme is added to the negative control.

In this application, singular terms such as “a” and “an” are to be interpreted to refer to one or more than one unless otherwise specified.

In one embodiment, the assay can be conducted in microliter quantities of bodily fluid, such as plasma, serum or other relevant fluids in a fluorescence-compatible microplate. The bodily fluid, fluorescently labeled donor, and acceptor are added to the wells such that the reaction mixtures contain undiluted amounts of bodily fluid. The well is incubated for sufficient time and at a temperature to convert substrate to product—in the case of CETP, this is typically about 90 minutes at 37° C. However, it is also possible to use initial velocities as a measure of activity or any intermediate value other than complete conversion, taking account of the kinetics of the reaction. The incubation time may thus be shortened in order to determine the initial velocity of the transfer reaction or measured before the transfer reaction comes to completion. At the desired end point, the fluorescence of both the test well and the negative control are read and the negative control fluorescence subtracted from that of the test well.

The inhibitor for the enzyme activity is, in one convenient embodiment, an antibody, although any other specific binding molecule, such as a peptidomimetic or an aptamer or a compound that inactivates the enzyme could also be used. As used in the present application, “antibody” refers not only to complete traditional antibody molecules, but also to fragments and to recombinantly produced forms such as single-chain Fv antibodies. As the assay is performed ex vivo, immunoreaction is not a concern, nevertheless, the antibodies may be chimeric or human or humanized if desired.

Depending on the enzyme to be analyzed, various inhibitors may well be known in the art. For example, other inhibitors of CETP activity are known such as torcetrapib, dalcetrapib, anacetrapib or evacetrapib.

The assay can be performed on bodily fluids of any subject. Humans are of the most interest, but other subjects that have circulatory systems containing the enzyme to be assayed may also be desirable. For example, in assessing possible treatments that modulate enzyme levels, laboratory animals such as mice, rats, rabbits and the like may be used. Veterinary uses are also contemplated for companion animals as well as agricultural livestock and animals useful in entertainment venues.

Biological fluids include blood, serum, plasma, semen, urine, cerebrospinal fluid, drainage fluids from wounds, saliva, digestive fluids or any other biological fluid which may contain an enzymatic activity of interest. Suitable fractions of any of these fluids could also be used. The improvement simply requires that the “blank” contain the same fluid as the test sample.

With respect to the remaining aspect of the invention regarding measuring enzyme activity by titration with serial dilutions of inhibitor. Undiluted samples as used in the improved assay are assessed for activity using various concentrations of the appropriate inhibitor by determining the concentration of inhibitor required to completely inhibit the enzyme. The enzyme activity in the sample can be calculated based on any known correspondence between the enzyme and its inhibitor. Thus, if the inhibitor is an antibody that has a 1:1 interaction with the enzyme, the concentration of inhibitor required to neutralize the activity in the sample will correspond to the concentration of enzyme in the sample.

The following examples are offered to illustrate but not to limit the invention.

An illustrative monoclonal antibody used in the example herein is designated TP2, a CETP neutralizing monoclonal antibody available from the University of Ottawa Heart Institute.

### Example 1

### Determination of CETP in Blood

Three plasma Samples (Z,N,6) were thawed at 25° C. and 100 μl of each was added to a fluorescence compatible microplate in triplicate×2. To one set of triplicates, 2 μl of 1 mg/ml TP2 in PBS was added (for the negative controls) and 2 μl of PBS was added to the other set of triplicates. A set of triplicate saline buffer blanks was also included on the microplate.

A mixture of neutral lipid donor particles (Reagent A) and acceptor particles (Reagent B) was made to create Reagent C according to the instructions provided with the commercially available kit from Roar Biomedical, Inc. (NY, N.Y.) catalog number RB-EVAK. Reagent C (5 μl) was added to each of the wells on the microplate, the plate was sealed with an adhesive aluminum plate sealer and placed in a 37° C. incubator for 90 minutes for an end-point assay.

The plate was read at 465 nm excitation and 535 nm emission, and the results are shown in Tables 1 and 2.

TABLE 1

| | | | | | | | Average | |
|---|---|---|---|---|---|---|---|---|
| | Raw Fluorescence Intensity Units | | | | | | −TP2 | +TP2 |
| | −TP2 mAb | | | +TP2 mAb | | | mAb | mAb |
| Plasma Z | 6984 | 7399 | 6552 | 3984 | 3703 | 3873 | 6978 | 3853 |
| Plasma N | 8374 | 8485 | 7698 | 4339 | 4771 | 4014 | 8186 | 4375 |
| Plasma 6 | 7189 | 8318 | 7037 | 3609 | 3727 | 3743 | 7515 | 3693 |
| Buffer | 3534 | 3562 | 3551 | 3767 | 3857 | 3530 | 3549 | 3718 |

TABLE 2
Transferred Fluorescence Intensity Units

| | Buffer as | TP2 mAb as | Δ TP2 mAb |
|---|---|---|---|
| | Negative Control | Negative Control | vs Buffer |
| Plasma Z | 3429 | 3125 | 304 |
| Plasma N | 4637 | 3811 | 826 |
| Plasma 6 | 3966 | 3822 | 144 |

In Table 2, the values in the first column of “Buffer as Negative Control” are obtained by subtracting the 3549 value for buffer from each of the average values labeled “−TP2 mAb” in Table 1. The values in Table 2 for the column labeled “TP2 mAb as Negative Control” are obtained by subtracting the value of the +TP2 mAb average in Table 1 from the −TP2 mAb average in Table 1 in each case. The third column in Table 2 shows that in each case, there was a difference in the values obtained using +TP2 mAb as a control as compared to using buffer. This difference is due to the matrix effect provided by the undiluted serum as an influence on the fluorescence units transferred. Plasma N in particular has a dramatic effect.

The matrix effect is accounted for when the mAb treated sample is used instead of the buffer.

1. An improved method for detecting the level of the activity of a desired enzyme in a bodily fluid of a subject by measuring the conversion of substrate to product in a reaction mixture comprising an undiluted amount of the bodily fluid, wherein the improvement comprises employing as a negative control a similar reaction mixture which further contains an inhibitor which is a binding or inactivating agent for said enzyme.

2. The method of claim 1 wherein said measuring is by determining the transfer of label from a donor substrate to an acceptor.

3. The method of claim 2 wherein label is a fluorescent label present as a quenched state in the donor substrate and as an unquenched state after transfer to acceptor.

4. The method of claim 1 wherein said inhibitor is an antibody.

5. The method of claim 1 wherein said undiluted amount is that wherein the bodily fluid comprises at least 50% of the reaction mixture.

6. The method of claim 5 wherein said undiluted amount is that wherein the bodily fluid comprises at least 85% of the reaction mixture.

7. The method of claim 6 wherein said undiluted amount is that wherein the bodily fluid comprises at least 90% of the reaction mixture.

8. The method of any of claims 1-7 wherein the desired enzyme is cholesteryl ester transfer protein (CETP), phospholipid transfer protein (PLTP), lipoprotein lipase (LPL), hepatic lipase (HL), hormone sensitive lipase (HSL), endothelial lipase (EL), phospholipase (PL), or lecithin:cholesterol acyl transferase (LCAT).

9. The method of claim 8, wherein the desired enzyme is CETP.

10. A kit for carrying out the method of claim 1 which kit comprises:
a container containing a substrate for an enzyme whose concentration is to be measured;
a container containing reagents for detection of the activity of the enzyme; and
a container containing a deactivating or binding agent for said enzyme.

11. The kit of claim 10 wherein the substrate for the enzyme comprises donor particles and receptor particles which are provided in separate containers.

12. The kit of claim 10 wherein the deactivator or binding agent is an antibody or fragment thereof.
